(19) United States
(12) Patent Application Publication
(10) Pub. No.: US 2024/0374266 A1
(43) Pub. Date: Nov. 14, 2024

(54) CATHETER DEFINING LONGITUDINALLY ARRANGED AND FLUIDICALLY CONNECTED OPENINGS AT A DISTAL REGION

(71) Applicant: Covidien LP, Mansfield, MA (US)

(72) Inventors: Tejashri Kumar, Fountain Valley, CA (US); Mark Ashby, Laguna Niguel, CA (US); Ya Guo, Irvine, CA (US); Jia Sun, Lake Forest, CA (US); Maria De Jesus Sanson, Mission Viejo, CA (US); Arshia Amin Ehsanipour, Portland, OR (US); Danyong Zeng, Aliso Viejo, CA (US)

(21) Appl. No.: 18/435,846

(22) Filed: Feb. 7, 2024

Related U.S. Application Data
(60) Provisional application No. 63/465,432, filed on May 10, 2023.

(51) Int. Cl. A61B 17/22; A61M 25/00

(52) U.S. Cl. CPC A61B 17/22; A61B 2017/22079; A61B 2017/22094; A61M 25/007

(57) ABSTRACT

Systems and methods for removing clots via aspiration are disclosed herein. A catheter includes an elongate body defining a lumen. The distal region includes a distal openings region, a proximal openings region and a thrombus interface region between the distal and the proximal openings regions. The distal and proximal openings regions each includes one or more openings in fluid communication with the lumen. In use, the catheter is positioned from a proximal side of the clot so that the distal openings region positioned at least partially distal of the clot and the proximal openings region positioned at least partially proximal of the clot. An aspiration catheter is positioned with its distal end at the proximal end of the clot. The clot is then withdrawn in the proximal direction with a combination of aspiration applied to one or more of the catheter or the aspiration catheter.

### CROSS-REFERENCE TO RELATED APPLICATION(S)

The present application claims priority to U.S. Provisional Patent Application No. 63/465,432, filed May 10, 2023, which is incorporated by reference herein in its entirety.

### TECHNICAL FIELD

The present technology relates to catheters having longitudinally arranged and fluidically connected openings at a distal end region. The present technology also relates to catheters having supporting structures for improved clot retrieval.

### BACKGROUND

Ischemic strokes are caused by interruption of the blood supply to the brain. For example, the blood supply may be interrupted by a thrombus (e.g., a blood clot) lodged in an artery responsible for feeding oxygenated blood to the brain. If the disruption in blood occurs for a sufficient amount of time, the continued lack of nutrients and oxygen causes irreversible cell death, potentially leading to permanent neurological deficit or death. Therefore, immediate restoration of blood flow is critical. One method of restoring blood supply to the brain involves removing the thrombus via mechanical thrombectomy, including stent-retriever thrombectomy and direct aspiration applied to the proximal end of the thrombus by the distal end of an aspiration catheter.

In order to restore blood supply in a timely manner, it has been found highly beneficial to fully remove the clot in an initial attempt, also referred to herein as a first pass. A first pass removal of a clot has been correlated to better clinical outcomes, and is referred to herein as the “first pass effect.” However, in certain instances, particularly when using an aspiration catheter, a clot may become fragmented or disengaged from the aspiration catheter during the initial attempt, thereby requiring additional passes. Accordingly, there is a need for systems, devices, and methods for addressing the problems noted above in order to increase the likelihood of achieving the first pass effect.

### SUMMARY

The present technology is illustrated, for example, according to various aspects described below. Various examples of aspects of the present technology are described as numbered clauses (1, 2, 3, etc.) for convenience. These are provided as examples and do not limit the present technology. It is noted that any of the dependent clauses may be combined in any combination, and placed into a respective independent clause. The other clauses can be presented in a similar manner.

Clause 1. A method for removing a clot from a vessel with a catheter and an aspiration catheter, wherein the catheter comprises: an elongate body defining a lumen extending between a proximal region and a distal region of the elongate body, wherein the distal region comprises a distal openings region, a proximal openings region and a thrombus interface region between the distal openings region and the proximal openings region, and wherein the distal openings region and the proximal openings region each comprise one or more openings in fluid communication with the lumen, the method comprising: positioning the catheter from a proximal side of the clot so that the distal region of the catheter extends through the clot with the distal openings region positioned at least partially distal of the clot and the proximal openings region positioned at least partially proximal of the clot; positioning an aspiration catheter distal end at the proximal end of the clot; and withdrawing the clot in the proximal direction with a combination of aspiration applied to one or more of the catheter or the aspiration catheter.

Clause 2. The method of any one of the Clauses herein, wherein the catheter is positioned outside of and parallel to the aspiration catheter.

Clause 3. The method of any one of the Clauses herein, wherein the aspiration catheter is positioned over the catheter.

Clause 4. The method of any one of the Clauses herein, wherein the aspiration catheter and the catheter are integrally formed as a single unit.

Clause 5. The method of any one of the Clauses herein, wherein positioning the catheter so that the distal region of the catheter extends through the clot with the distal openings region positioned distal of the clot and the proximal openings region positioned proximal of the clot cause the pressure differential between the proximal side of the clot and the distal side of the clot to be reduced.

Clause 6. The method of any one of the Clauses herein, wherein during withdrawing of the clot, the position of the catheter relative to the clot with the distal openings region positioned distal of the clot and the proximal openings region positioned proximal of the clot maintained in order to maintain the reduce pressure differential.

Clause 7. The method of any one of the Clauses herein, wherein withdrawing the clot in the proximal direction comprising applying aspiration to both the catheter and the aspiration catheter.

Clause 8. The method of any one of the Clauses herein, wherein aspiration is applied first to the catheter and then to the aspiration catheter.

Clause 9. The method of any one of the Clauses herein, wherein aspiration is applied first to the aspiration catheter and then to the catheter.

Clause 10. The method of any one of the Clauses herein, wherein aspiration is applied simultaneously to the catheter and to the aspiration catheter.

Clause 11. The method of any one of the Clauses herein, wherein aspiration is applied to the aspiration catheter and not to the catheter.

Clause 12. The method of any one of the Clauses herein, wherein aspiration is applied to the catheter and not to the aspiration catheter.

Clause 13. The method of any one of the Clauses herein, wherein the catheter comprises a supporting structure housed in the elongate body.

Clause 14. The method of any one of the Clauses herein, wherein the supporting structure is disposed in a sidewall of the elongate body at a position radially opposite one or more openings of one or more of the proximal openings region and the distal openings region.

Clause 15. The method of any one of the Clauses herein, wherein the supporting structure has a greater stiffness than the elongate body.

Clause 16. The method of any one of the Clauses herein, wherein the elongate body comprises one or more ridges integrally formed thereon.

Clause 17. A method for removing a clot from a vessel with a catheter and an aspiration catheter, wherein the catheter comprises: an elongate body defining a lumen extending between a proximal region and a distal region of the elongate body, wherein the distal region comprises a thrombus interface region, wherein the thrombus interface region comprises one or more openings in fluid communication with the lumen, the method comprising: positioning the catheter from a proximal side of the clot so that the distal region of the catheter extends through the clot with the openings of the thrombus interface region positioned within the clot; positioning an aspiration catheter distal end at the proximal end of the clot; and withdrawing the clot in the proximal direction with a combination of aspiration applied to one or more of the catheter or the aspiration catheter.

Clause 18. The method of any one of the Clauses herein, wherein the catheter is positioned outside of and parallel to the aspiration catheter.

Clause 19. The method of any one of the Clauses herein, wherein the aspiration catheter is positioned over the catheter.

Clause 20. The method of any one of the Clauses herein, wherein the aspiration catheter and the catheter are integrally formed as a single unit.

Clause 21. The method of any one of the Clauses herein, wherein positioning the catheter so that the distal region of the catheter extends through the clot with a distal openings region positioned distal of the clot and a proximal openings region positioned proximal of the clot cause the pressure differential between the proximal side of the clot and the distal side of the clot to be reduced.

Clause 22. The method of any one of the Clauses herein, wherein withdrawing the clot in the proximal direction comprises applying aspiration to both the catheter and the aspiration catheter.

Clause 23. The method of any one of the Clauses herein, wherein the catheter comprises a supporting structure housed in the elongate body.

Clause 24. The method of any one of the Clauses herein, wherein the supporting structure is disposed in a sidewall of the elongate body at a position radially opposite one or more openings of one or more of a proximal openings region and a distal openings region.

Clause 25. A method for removing a clot from a vessel with a catheter, wherein the catheter comprises: an elongate body extending between a proximal region and a distal region, the elongate body defining a first lumen having a distal terminus and a second lumen extending to the distal region, wherein the distal region comprises a thrombus interface region, wherein the distal region comprises a distal openings region, a proximal openings region and a thrombus interface region between the distal openings region and the proximal openings region, wherein the distal openings region and the proximal openings region each comprise one or more openings in fluid communication with the second lumen the method comprising: positioning the catheter from a proximal side of the clot so that the distal region of the catheter extends through the clot with the openings of the thrombus interface region positioned within the clot and the distal terminus of the first lumen is positioned on a proximal side of the clot; and withdrawing the clot in the proximal direction with a combination of aspiration applied to one or more of the first lumen or the second lumen.

Clause 26. A device for removing a clot from a vessel comprising: an elongate body extending between a proximal region and a distal region; a first lumen defined by the elongate body, the first lumen having a distal terminus; a second lumen defined by the elongate body; a distal openings region in the distal region; a proximal openings region in the distal region; and a thrombus interface region between the proximal openings region and the distal openings region, wherein the proximal openings region and the distal openings region each comprises one or more openings in fluid communication with the second lumen.

Clause 27. A device for removing a clot from a vessel comprising: an elongate body defining a lumen extending between a proximal region and a distal region of the elongate body; a proximal openings region in the distal region; a distal openings region in the distal region at a position distal to the proximal openings region; and a thrombus interface region between the proximal openings region and the distal openings region, wherein the proximal openings region and the distal openings region each comprise one or more openings in fluid communication with the lumen.

Clause 28. The device of any one of the Clauses herein, further comprising a supporting structure housed in the elongate body.

Clause 29. The device of any one of the Clauses herein, wherein the supporting structure spans the entirety of the elongate body from the proximal region to the distal region.

Clause 30. The device of any one of the Clauses herein, wherein the supporting structure is disposed in a sidewall of the elongate body at a position radially opposite one or more openings of one or more of the proximal openings region and the distal openings region.

Clause 31. The device of any one of the Clauses herein, wherein the supporting structure has a greater stiffness than the elongate body.

Clause 32. The device of any one of the Clauses herein, wherein the supporting structure extends radially outwardly from a sidewall of the elongate body.

Clause 33. The device of any one of the Clauses herein, wherein the lumen is non-concentric.

Clause 34. The device of any one of the Clauses herein, wherein the elongate body comprises one or more ridges integrally formed thereon.

Clause 35. The device of any one of the Clauses herein, wherein at least one of the one or more ridges is an internal ridge.

Clause 36. The device of any one of the Clauses herein, wherein at least one of the one or more ridges is an external ridge.

Clause 37. The device of any one of the Clauses herein, wherein the device is configured to be coupled to an aspiration catheter.

Clause 38. A system for removing a clot from a vessel, the system comprising: an aspiration catheter; and a catheter comprising: an elongate body defining a lumen extending between a proximal region and a distal region of the elongate body, a proximal openings region in the distal region, a distal opening regions in the distal region, and a thrombus interface region between the proximal openings region and the distal openings region, wherein the distal openings region and the proximal openings region each comprise one or more openings in fluid communication with the lumen.

Clause 39. The system of any one of the Clauses herein, wherein the catheter is positioned outside of and parallel to the aspiration catheter.

Clause 40. The system of any one of the Clauses herein wherein the aspiration catheter is positioned over the catheter.

Clause 41. The system of any one of the Clauses herein, wherein the aspiration catheter and the catheter are integrally formed as a single unit.

Clause 42. The system of any one of the Clauses herein, wherein the catheter further comprises a supporting structure housed in the elongate body.

Clause 43. The system of any one of the Clauses herein, wherein the supporting structure has a greater stiffness than the elongate body.

Clause 44. The system of any one of the Clauses herein, wherein the supporting structure is disposed in a sidewall of the elongate body at a position radially opposite one or more openings of one or more of the proximal openings region and the distal openings region.

Clause 45. The system of any one of the Clauses herein, wherein the elongate body comprises one or more ridges integrally formed thereon.

Claim 46. The system of any one of the Clauses herein, wherein the thrombus interface region is devoid of openings.

Clause 47. The system of any one of the Clauses herein, wherein the openings comprise discrete apertures formed in the sidewall of the elongate body.

Clause 48. The system of any one of the Clauses herein, wherein the openings comprise contiguous voids in the sidewall of the body.

Clause 49. The system of any one of the Clauses herein, further comprising a suction source configured to be fluidically coupled to at least one of the aspiration catheter or the catheter.

Additional features and advantages of the present technology are described below, and in part will be apparent from the description, or may be learned by practice of the present technology. The advantages of the present technology will be realized and attained by the structure particularly pointed out in the written description and claims hereof as well as the appended drawings.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 shows a blood vessel occluded by a blood clot, and depicts the pressures and forces associated with the blood supply and the blood clot.

FIG. 2A shows a blood clot causing an occlusion in a blood vessel.

FIGS. 2B-2C show corking of a blot clot in an aspiration catheter.

FIGS. 3A-3I show a catheter with distally arranged openings, according to embodiments of the disclosed technology.

FIGS. 4A-4E show steps of a thrombectomy with a system including a catheter having distally arranged openings and an aspiration catheter, in accordance with embodiments of the disclosed technology.

FIGS. 5A and 5B show a catheter with distally arranged openings integrated with an aspiration catheter, according to embodiments of the disclosed technology.

FIGS. 6A-6C show steps of a thrombectomy in tortuous anatomy with a system including a catheter with distally arranged openings and an aspiration catheter, in accordance with embodiments of the disclosed technology.

FIGS. 7A-7D show steps of a thrombectomy with a system including aspirating with a catheter with distally arranged openings and aspirating with an aspiration catheter, in accordance with embodiments of the disclosed technology.

FIGS. 8A-8C show steps of a thrombectomy with a system including aspirating with a catheter with distally arranged openings and aspirating with an aspiration catheter, in accordance with embodiments of the disclosed technology.

FIG. 9 shows a distal region of a catheter including ridges, according to embodiments of the disclosed technology.

FIG. 10 shows a distal region of a catheter with distally arranged openings and further including an atraumatic tip according to embodiments of the disclosed technology.

FIG. 11 shows a cross-section of an example catheter with distally arranged openings and further including a supporting structure, in accordance with embodiments of the disclosed technology.

FIG. 12 shows a cross-section of an example catheter with distally arranged openings and further including a supporting structure, in accordance with embodiments of the disclosed technology.

FIG. 13 shows a cross-section of an example catheter with distally arranged openings and further defining a non-concentric lumen, in accordance with embodiments of the disclosed technology.

FIG. 14 shows a cross-section of an example catheter with distally arranged openings and further including a ridge structure, in accordance with embodiments of the disclosed technology.

FIG. 15 shows a side view of an example catheter with a reduced-wall thickness region and distally arranged openings, in accordance with embodiments of the disclosed technology.

FIG. 16 shows a cross-section of an example catheter with distally arranged openings, in accordance with embodiments of the disclosed technology.

FIG. 17 shows a cross-section of an example catheter with conical or flared arranged openings, in accordance with embodiments of the disclosed technology.

### DETAILED DESCRIPTION

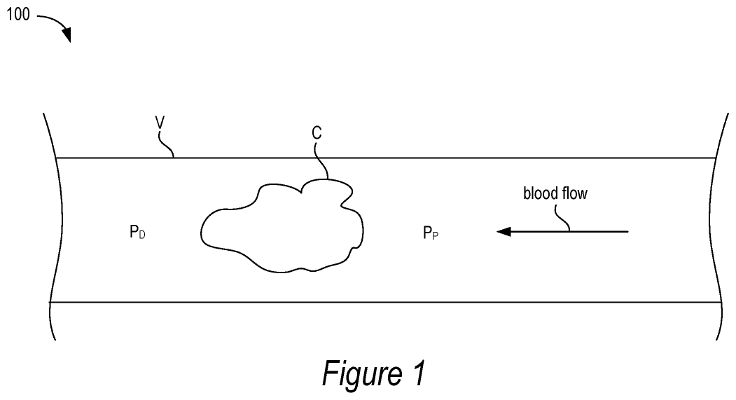

The present technology relates to systems, devices, and methods for treating vascular obstructions, such as vessel occlusions. In some embodiments, for example, a device includes a catheter having distally arranged openings. In some embodiments, the catheter includes one or more supporting structures to assist in navigating of the catheter through tortuous vasculature. Specific details of several embodiments of the technology are described below with reference to FIGS. 1-17.

The detailed description set forth below is intended to describe various configurations of the subject technology and is not intended to represent the only configurations in which the subject technology may be practiced. The detailed description includes specific details for the purpose of providing a thorough understanding of the subject technology. However, it will be clear and apparent to those skilled in the art that the subject technology is not limited to the specific details set forth herein and may be practiced using one or more implementations.

As noted previously, when attempting to remove a blood clot to restore blood supply, it is generally advantageous to fully remove the clot in the first pass. One obstacle faced by current direct aspiration thrombectomy methods applied at the proximal end of the clot, which may prevent achieving a first pass effect, is an existing pressure differential between proximal and distal sides of the clot. For example, as shown in FIG. 1, the pressure P_{P} on the proximal side of the clot C is greater than the pressure P_{D} on the distal side of the clot (Pₚᵣₒₓᵢₘₐₗ>P_{distal}). The resulting pressure gradient creates forces against the clot in the distal direction. In order for the clot to be removed from a vessel in a proximal direction via direct aspiration thrombectomy, both the forces of the vessel walls interacting with the clot and the forces caused by the pressure gradient need to be overcome. Unfortunately, overcoming these forces using only direct aspiration applied at the proximal end of the clot may lead to fragmentation or breaking up of the clot, which may necessitate additional passes to remove all portions of the clot and restore blood supply. This fragmentation also risks portions of the clot migrating distally downstream, which may lead to embolization at more distal locations within the vessel.

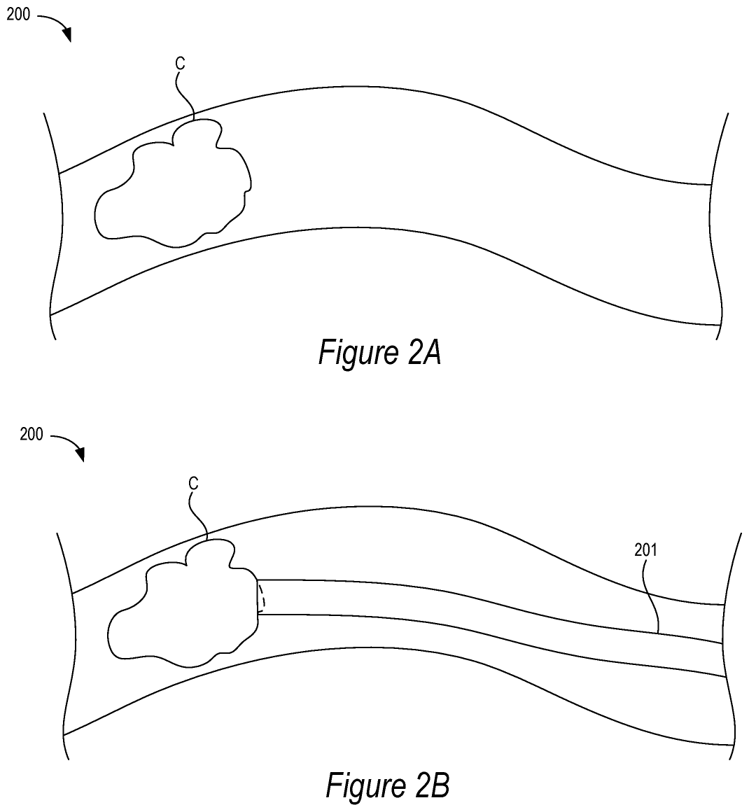

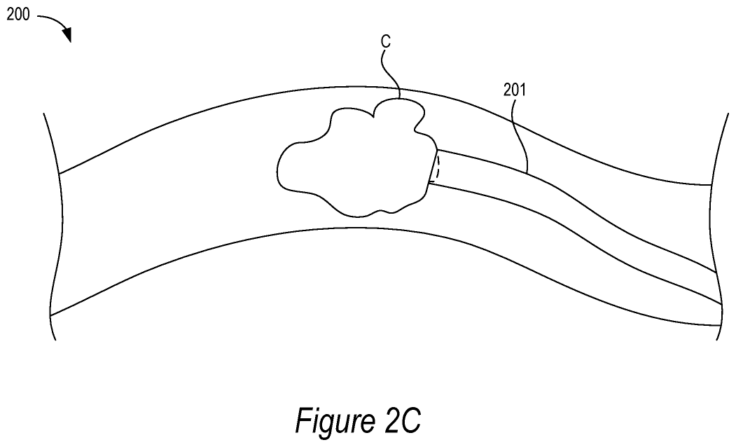

Another problem with current direct aspiration thrombectomy methods relates to navigation through tortuous anatomy. For example, current direct aspiration methods use catheters with large bores (e.g., catheters having an inner diameter equal to or greater than 0.068″). Due to their large size and stiffness, such catheters may be challenging to navigate to the middle or distal anatomies (MEVO/DEVO). Another navigation issue with current direct aspiration thrombectomy methods relates to fibrin rich clots which may obstruct the tip of the aspiration catheter and block aspiration. For example, as shown in FIGS. 2A and 2B, during direct aspiration, the proximal end of the clot C may be aspirated into the inside of the distal end of the aspiration catheter 201 and clog the distal opening of the aspiration catheter 201, leaving the distal end of the clot outside of the aspiration catheter 201 and thus unsecured. This phenomenon may be referred to as “corking.” Removing a corked clot in the distal end of an aspiration catheter may involve retracting the aspiration catheter through tortuous anatomy, as shown, for example, in FIG. 2C, which may lead to clot fragmentation and/or losing part of the clot from the unsecured distal end of the clot into a distal territory, which as previously noted, is undesirable as it may necessitate additional passes. Further, the entire clot may dislodge from the distal end of the catheter during retraction of the aspiration catheter. Suppose the clots are heterogeneous (e.g., soft and firm). In that case, when retrieving the clots, the softer part of the clots can easily get dislodged to the distal territories around the bifurcations or tortuosity.

Partial or complete loss of the blood clot during retraction of the aspiration catheter can necessitate further access to the treatment site, creating significant delays in the procedure as the system must be renavigated to the treatment site(s) to make a subsequent pass and additionally creates added trauma to the arteries through which the aspiration catheter is again or newly passed.

Embodiments of the present technology relate to systems for equalizing the pressure on the distal and proximal sides of clots. In various implementations, the systems can be made from metals such as Nitinol or stainless steel or polymer such as silicone with various durometers with or without radiopaque material such as barium sulphate and tungsten embedded for fluoroscopic enhancement. This can be used as a standalone device as an accessory or in conjunction with another device, such as a catheter.

The systems may include catheters defining a lumen and longitudinally spaced apart openings in fluid communication with the lumen. The longitudinal spacing between at least two of the openings allows for the at least one opening to be positioned on opposite sides of the clot, e.g., a distal side and a proximal side, in order to reduce the pressure differential between the distal and proximal sides of the clot, as will be discussed in greater detail below. By thus reducing this pressure differential, the applied suction required to successfully pull the clot into an aspiration catheter can be reduced, thereby increasing the likelihood of successful first pass clot removal.

### I. EXAMPLE CATHETERS HAVING DISTALLY ARRANGED OPENINGS

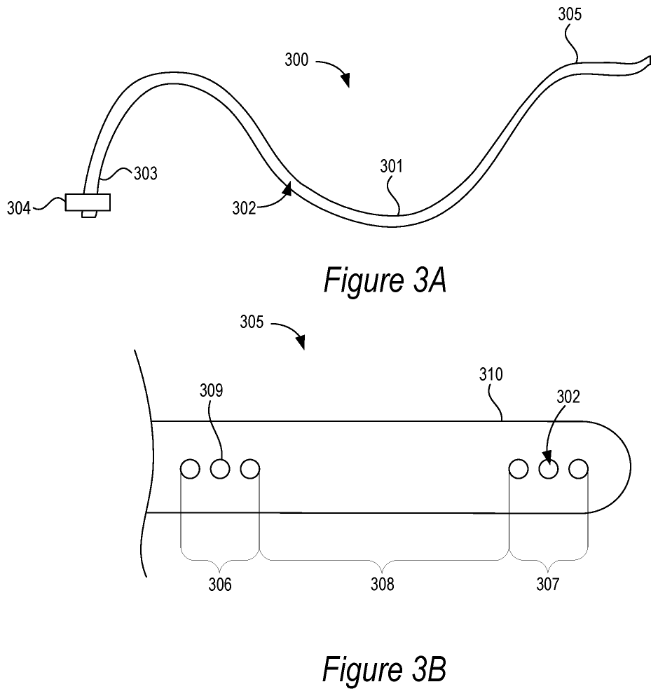

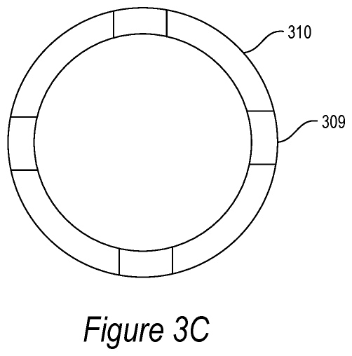

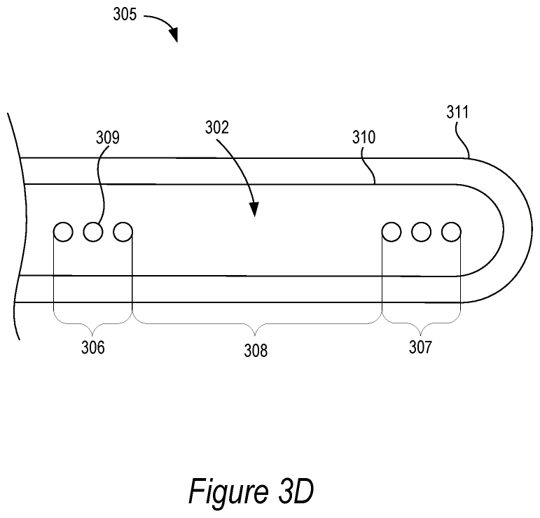

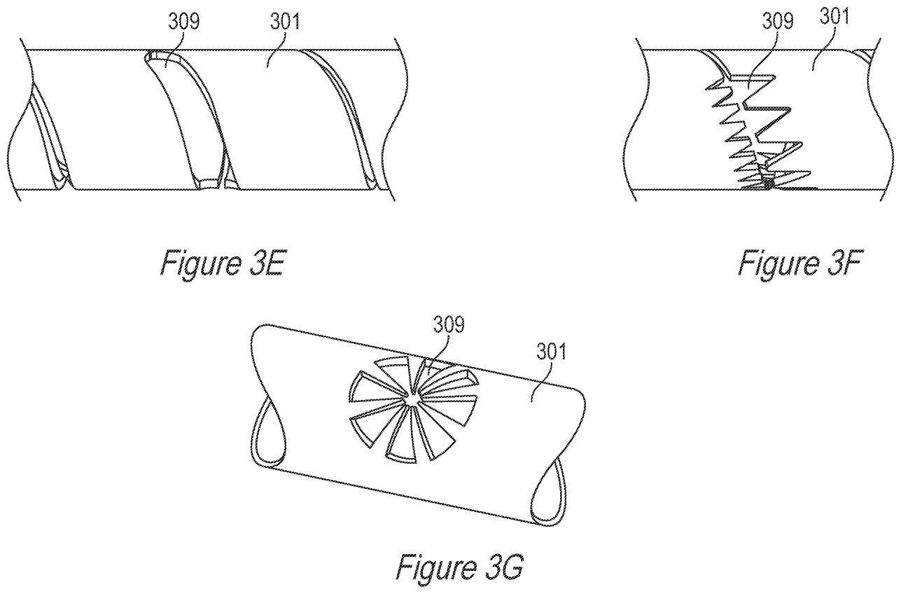

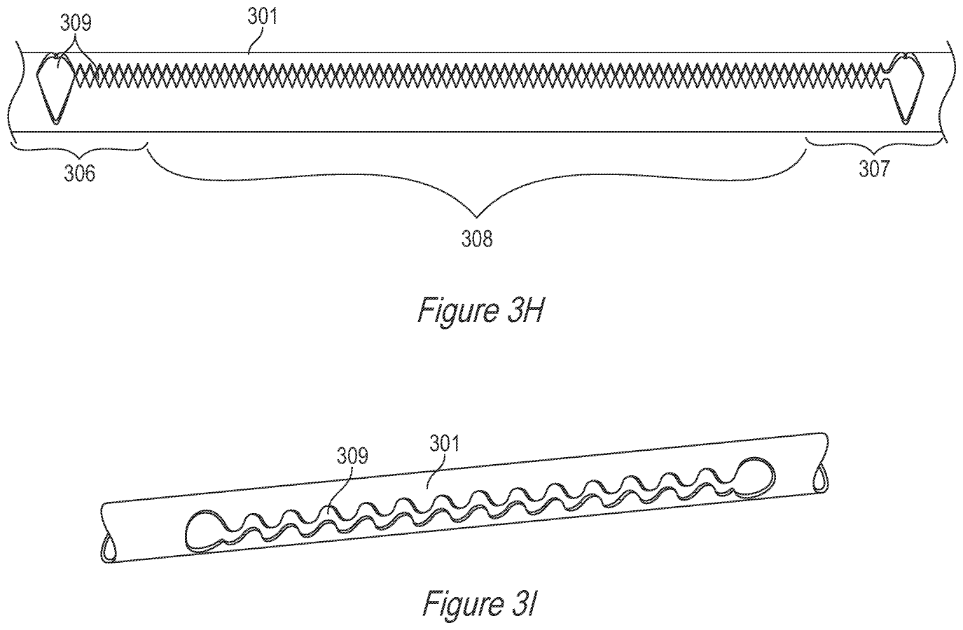

FIG. 3A shows an example of a catheter 300 which may be included in systems for reducing a pressure differential across distal and proximal sides of clots. The catheter 300 may include an elongate body 301 defining a lumen 302. The lumen 302 may have any suitable inner diameter. Among examples, the inner diameter can be between 0.010 inch and 0.045 inch. The elongate body 301 may be formed of metals, polymers, or a combination thereof. The elongate body 301 may define a proximal region 303 to remain outside of the body during procedures. In some embodiments, the proximal region 303 may include a coupling 304 for fluidically connecting to a fluid source and/or a vacuum source. The length and diameter of the catheter 300 are suitable for inserting into a human patient and capable of reaching a target treatment site (e.g., a location of a target embolus (blood clot) in the region above the subclavian and common carotid arteries) while still being accessible to a clinician from outside a patient's body. Blood clots in distal intracranial territories may require catheters longer than typical 145 cm-160 cm microcatheters, such as 170 cm or longer.

As shown in FIG. 3A, the catheter 300 may include a distal region 305 opposite the proximal region 303. FIG. 3B shows a detailed view of an embodiment of a distal region 305. In some embodiments, the distal region 305 may include a proximal openings region 306, distal openings region 307, and a thrombus interface region 308. As shown, the thrombus interface region 308 may be located between the proximal openings region 306 and the distal openings region 307, wherein the distal openings region 307 is located distal of the thrombus interface region 308. In some implementations, the thrombus interface region 308 may at least partially overlap the proximal openings region 306 and/or the distal openings region 307.

Each of the proximal openings region 306 and the distal openings region 307 defines one or more openings 309 extending through a wall 310 of the elongate body into a central lumen 302 defined by the elongate body. In some embodiments, each of the proximal openings region and the distal openings region may define a plurality of openings. In various examples, the openings 309 can take the form of windows, apertures, voids, cuts, or other such structures that allow fluid to pass therethrough. The plurality of openings of each region may be arranged with longitudinal spacing and/or radial spacing. For example, as shown in FIG. 3B, each of the proximal openings region and the distal openings region can define three longitudinally arranged openings on one radial side of the elongated body. Each longitudinally arranged opening may be an opening of a radial row of openings or may be a single opening. For example, FIG. 3C shows a cross-section of a portion of the elongate body defining a radial row of openings, and specifically shows a row of 4 radially arranged openings that are distributed circumferentially about the wall 310 of the catheter 300. Each radial row of openings may include any number of openings, including, but not limited to, one opening or four openings, as is shown in FIG. 3C.

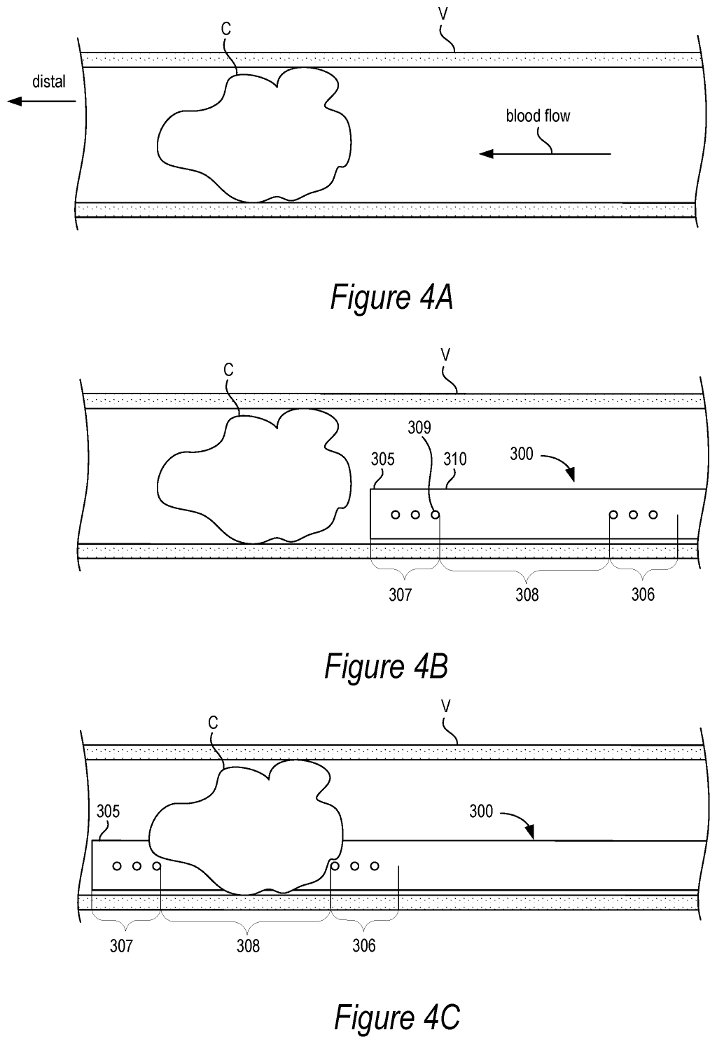

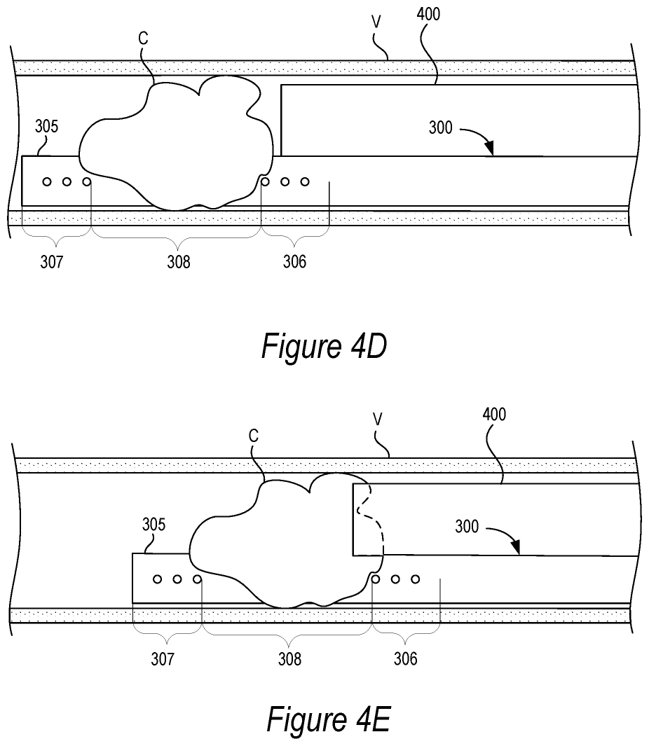

The openings of the proximal openings region and the openings of the distal openings region may be in fluid communication with each other via the central lumen 302, as is shown in the cross-sectional view of FIG. 3D. Due to the fluid communication, pressure external to the distal openings region may be brought toward equalization with pressure external to the proximal openings region, as will be discussed in greater detail in relation to FIGS. 4A-4E.

In some embodiments, the distal end 311 of the elongate body may have a closed end, for instance comprising a rounded shape so as to form an atraumatic tip. A rounded distal end can facilitate distal navigation of the catheter without catching corners against a blood vessel wall. In some embodiments, the distal end may be open, and may define an opening of the distal openings region, used for pressure equalization and/or aspiration. In some embodiments, an open distal end may define the sole opening of a distal openings region. In some embodiments, the distal end may include a valve allowing for a guidewire to extend through the central lumen, wherein the valve is closed if no guidewire is present or the valve may only be closed when a guidewire is present.

As shown in FIGS. 3B and 3D, the thrombus interface region 308 may be a portion of the elongate body between the proximal openings region 306 and the distal openings region 307. In some embodiments, for example as shown in FIGS. 3B-3D, the thrombus interface region 308 may not include any openings. In some embodiments, the thrombus interface region 308 may include one or more openings. Openings in the thrombus interface region may be used for pressure equalization and/or aspiration, as will be discussed in greater detail below. The thrombus interface region may be sized to correspond to the length of a thrombus that the catheter is used in the removal of. In some embodiments, the thrombus interface region may be between 5 mm and 40 mm in length. As will be discussed in greater detail below in relation to FIGS. 4A-4E, when in use, the thrombus interface region is positioned through or radially adjacent to a thrombus such that the proximal openings region and the distal openings region are positioned on opposite ends of the thrombus and each include at least one opening not obstructed by the thrombus.

In certain embodiments, the distal end may feature a valve designed to permit the extension of a guidewire through the central lumen, aiding navigation during the procedure. The valve is designed to allow the passage of a guidewire without any leakage when the guidewire is present. Alternatively, the valve can remain closed in the absence of a guidewire or may only close when a guidewire is introduced.

In some embodiments, the openings of the proximal openings region, the distal openings region, and/or the thrombus interface region, may be circular. For example as shown in FIG. 3D, the openings in the proximal openings region and distal openings region are circular. In some embodiments, the openings may be sized to prevent ingestion of the thrombus into the central lumen, and may be sized between 5% and 100% of the OD of the catheter 300. In some implementations, the openings may each have an opening size no more than about 90%, 80%, 70%, 60%, 50%, 40%, 30%, 20%, or 10% of the OD of the catheter 300. Among various examples, the OD of the catheter may range between about 0.010 in to 0.050 in.

FIGS. 3E-3I illustrate additional configurations of the opening(s) 309 of the catheter 300. As illustrated in FIG. 3E, the catheter body 301 can include a helical or spiral cut, with some or all of the spiral cut defining one or more openings 309. In some implementations, the opening(s) 309 take the form of widened portions of the spiral cut, in which the open space of the spiral cut is larger than in adjacent regions of the spiral cut. Optionally, the spiral cut (or other suitable cut pattern) extends along some or substantially all of the length of the catheter 300, and may be tailored (e.g., by selecting and/or varying pitch, cut size, length, etc.) to provide the desired mechanical characteristics for the catheter 300 (e.g., column strength, flexibility, kink-resistance, etc.).

The laser-cutting pattern's role in customizing the catheter's stiffness gradient from distal to proximal can provide significant benefits. For instance, this may ensure that the catheter is tailored to meet the specific demands of each procedure, optimizing its delivery capabilities and allowing it to reach the farthest sections of the vessel with precision and safety. The slot-shaped open design can also be implemented to create an opening structure along the spiral cut feature, which serves to minimize the opening's impact on the stiffness introduced by the spiral cut. This design ensures that the device maintains a smooth stiffness transition from the distal to proximal end after adding those local aspiration opening.

In the example illustrated in FIG. 3F, the opening 309 is defined by a saw-tooth opening that is contiguous with a spiral cut extending along the body 301 of the catheter 300. FIG. 3G illustrates yet another configuration, in which the opening 309 takes the form of a star-like shape. In some instances, it may be beneficial to provide a given overall open volume (thereby allowing a determined amount of fluid flow therethrough) while utilizing geometrical features to reduce the amount of contiguous open space. For instance, the star-like shape of FIG. 3G may have a similar total open area to a simple circular opening, yet the star-like opening of FIG. 3G may be less likely to permit an internal guidewire to pass therethrough because the open areas are segmented.

As depicted in FIGS. 3H and 3I, the opening 309 can form a continuous void that spans across a length of the catheter 300, for instance extending across a proximal openings region 306, a thrombus interface region 308, and a distal openings region 307. In some implementations, the continuous void can include sub-openings that are contiguously joined together. In the example depicted in FIG. 3H, the opening 309 takes the form of a sinusoidal, undulating, or serpentine void that extends across a length of the catheter 300. In these and other examples, the size, shape, and configuration of the opening(s) 309 can be selected to achieve the desired mechanical characteristics (e.g., stiffness and/or flexibility of the catheter 300) as well as the desired fluid dynamics (e.g., permitting a given fluid flow rate therethrough).

These various configurations of openings (e.g., star, sinusoidal or saw-style) can serve a beneficial role during the aspiration procedures, particularly in clot retrieval. These specialized structural features can be configured to create localized deformations or irregularities in the device's surface. This design strategy could offer several significant advantages. First, the localized deformations and irregularities in the device's surface can enhance blood clot engagement. By creating areas of increased contact and pressure, the device can effectively “lock onto” the clot, significantly enhancing the chances of successfully capturing and removing it. Second, during vacuum or aspiration processes, these surface features can help secure the clot within the device's working end. The increased contact area and localized deformations act as anchors, preventing the clot from dislodging or escaping during the aspiration process. This ensures thorough and efficient clot removal.

FIGS. 4A-4E show an example method of a system for removing a clot C from a vessel V. As shown in FIG. 4A a clot C is lodged against the walls of a vessel V. As discussed above, the pressure on the proximal side of the clot C (right side of FIGS. 4A-4E), is greater than the pressure on the distal side of the clot (left side of FIGS. 4A-4E), thus causing a force on the clot in the distal direction. As noted above, the use of a catheter 300 as described herein can reduce the pressure differential across the clot C, thereby facilitating removal of the clot C via an aspiration catheter 400.

In some embodiments, a catheter, for example catheter 300 as shown in FIG. 3A, may be introduced in the vasculature, and the distal region may be navigated to the proximal side of the clot C, for example, as shown in FIG. 4B. In some embodiments, the catheter 300 may be introduced into the vasculature through a larger catheter, for example a guide catheter or an aspiration catheter. The distal region 305 of the catheter 300 may be advanced distally through the clot C and/or between the clot C and the vessel wall, for example as shown in FIG. 4C. The distal region 305 may be advanced so that one or more of the openings 309 in the distal openings region 307 are distal of the clot and one or more of the openings in the proximal openings region 306 are proximal of the clot, so that the pressure differential between the proximal side and distal side of the clot is reduced via the fluid communication of the openings distal and proximal to the clot. In this position, at least some fluid flow in the proximal direction may pass into the lumen of the catheter 300 via the openings 309 in the proximal openings region 306, and then may optionally pass out of the lumen of the catheter 300 and into the vessel V through the openings 309 in the distal openings region 307. As such, some fluid flow bypasses the clot C through the lumen of the catheter 300. This bypass fluid flow results in a decrease or elimination of the pressure differential within the vessel V between the region distal to the clot C and the region proximal to the clot C.

With the pressure differential between the proximal side and distal side of the clot reduced with the catheter 300 in place, the clot may be removed via mechanical thrombectomy, for example, but not limited to, direct aspiration. In some embodiments, an aspiration catheter 400 may be introduced into the vasculature and navigated so that the distal end of the aspiration catheter 400 is adjacent to the proximal end of the clot C. For example, as shown in FIG. 4D, the aspiration catheter 400 may be introduced parallel to the catheter 300. In some embodiments, the aspiration catheter 400 may be introduced over the catheter 300, such that the catheter 300 is at least partially received within a lumen of the aspiration catheter 400. In certain embodiments, the clot (C) will be firmly secured in place on both the distal and proximal ends through the aspiration hole in the catheter 300.

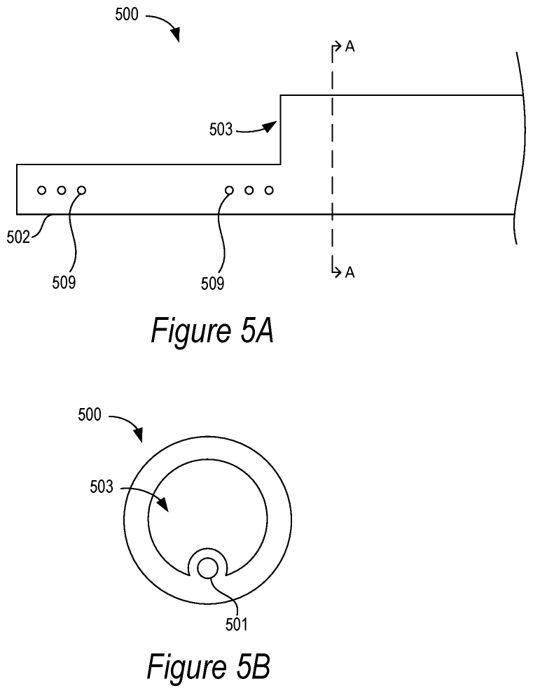

In some embodiments, a catheter 300 and the aspiration catheter 400 may be formed with an integral distal end assembly, as shown for example in FIGS. 5A and 5B. As shown, a combination catheter 500 may include a first lumen 501 in fluid communication with a distal region 502. Distal region 502 may be structurally the same as distal region 305 of catheter 300 described in relation to FIGS. 3A-3D. For instance, the first lumen 501 can be in fluid communication with the openings 509 in the distal region 502. Combination catheter 500 may also include a second lumen 503, which can be larger than the first lumen 501 and configured to receive a blood clot therein. Second lumen 503 may be used for direct aspiration similarly to aspiration catheter 400 in the method shown in FIGS. 4A-4E.

With a distal region 305/502 of a catheter positioned to reduce the pressure differential between the proximal side and distal side of the clot, as shown for example in FIG. 4D, the aspiration catheter 400 or second lumen 503 is able to engage the proximal end of the clot and the clot is able to be withdrawn in the proximal direction by the aspiration catheter and catheter combination without the previously present pressure force attributable to the high pressure differential across the clot C.

In some embodiments, for example as shown in FIG. 4E, the catheter 300 may be withdrawn concurrently with the aspiration catheter 400 in order to maintain the position of the openings 309 relative to the clot C so as to maintain the reduced pressure differential between the proximal side and distal side of the clot C. The initial and continuous reduced pressure differential between the proximal side and distal side of the clot reduces the likelihood of clot fragmentation, relative to direct aspiration without the reduced pressure differential between the proximal side and distal side of the clot, and therefore increase the likelihood of achieving a first pass effect.

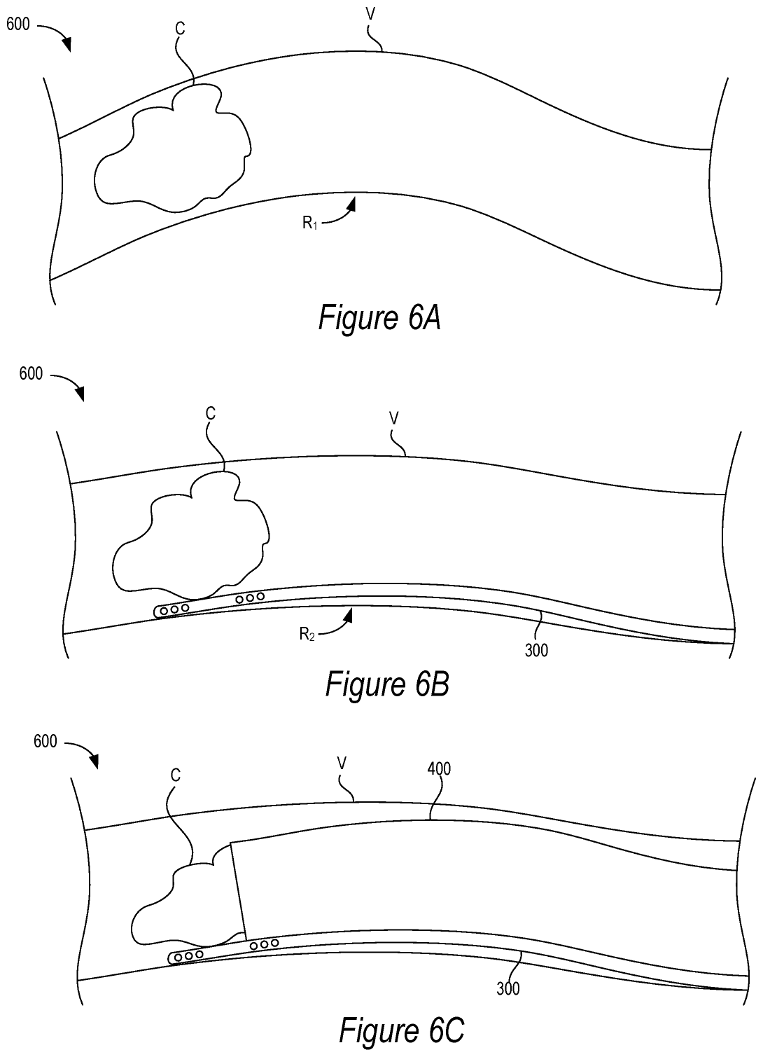

In addition to pressure equalization, in some embodiments, the catheter 300 may also provide the advantage of straightening the vessel prior to introducing the aspiration catheter 400, during initial aspiration by the aspiration catheter 400, and/or during withdrawal of the aspiration catheter 400. Such straightening of the vessel is particularly beneficial in cases with the clot positioned within or immediately distal of a tortuous anatomy, for example as shown in FIG. 6A. Similarly to FIGS. 4B and 4C, a catheter 300 may be introduced through a clot, or alongside the clot as is shown in FIG. 6B. Further, as shown in FIG. 6B, relative to FIG. 6A, the catheter 300 causes the radius of the bend to increase from R₁ to R₂, or in other words straightens the vessel. With the vessel in a more straight arrangement, the aspiration catheter 400 may be introduced with the distal end interfacing the proximal end of the clot C in a more direct line than if attempted to reach the clot without straightening, for example with the vessel radius as shown in FIG. 6A.

In addition to straightening the vessel for initial positioning of the aspiration catheter, as discussed above, the catheter 300 may be withdrawn concurrently with the aspiration catheter 400. During withdrawal, the distal openings region 307 remains distal of the clot and locally causes the portion of the vessel through which the clot C is transversed to be straightened. This local straightening reduces interference between the clot C and the vessel wall, and therefore reduces the likelihood of fragmentation. Further, the portion of the catheter 300 crossing the clot C reduces the circumferential contact of the clot C in contact with the vessel wall and accordingly reduces friction of the vessel wall opposing proximal movement of the clot C.

In some embodiments, during thrombectomies, a catheter, for example catheter 300 as shown in FIGS. 3A-3D or a similar catheter, is provided with openings along the thrombus interface region 308. In addition or alternatively to providing the reduced pressure differential between the proximal side and distal side of the clot, the catheter 300 may also be used for other uses, including, but not limited to, providing vacuum/aspiration and/or delivering medicament therethrough. In some embodiments, the catheter 300 may be used for aspiration, and the aspiration catheter may concurrently and/or subsequently be used for aspiration, and both the catheter 300 and aspiration catheter 400 may be withdrawn concurrently. Drawing a vacuum in the lumen of the catheter 300 may be used to apply a suction force to internal portions or external portions of the clot C adjacent to the openings of the thrombus interface region 308, thereby providing the ability to apply axial force on the clot using the catheter 300. In some embodiments, the aspiration catheter 400 may initially be used for aspiration, followed by aspiration by the catheter 300 and both the catheter 300 and aspiration catheter 400 may be withdrawn concurrently.

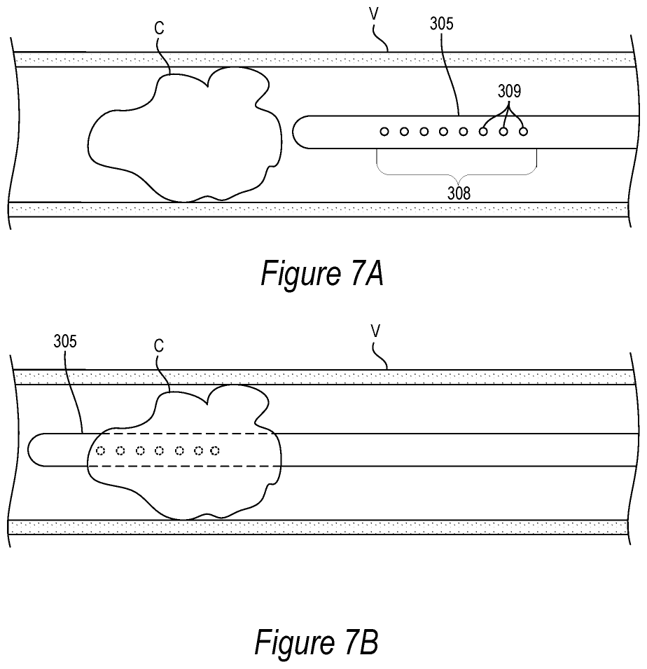

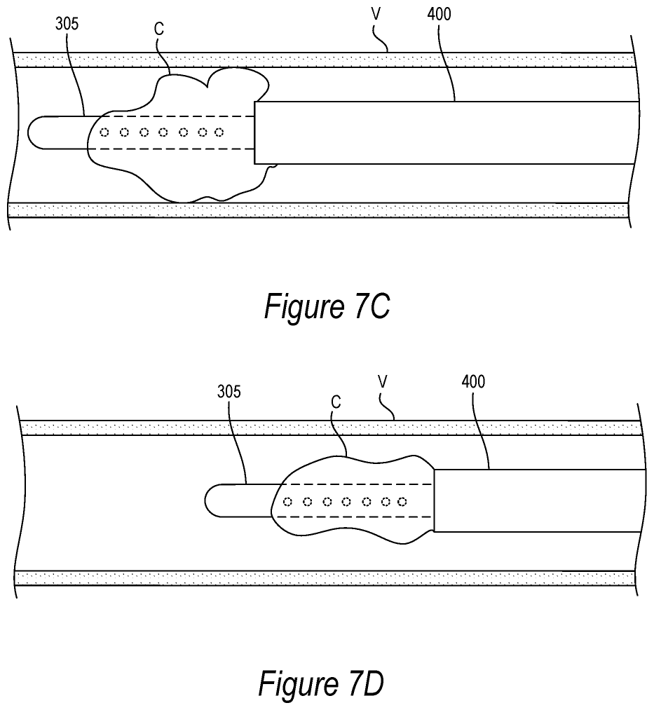

FIGS. 7A-7D show an example method for removing a clot C from a vessel V. While stenting has become commonplace in instances in which vessel straightening occurs within the distal anatomies as a consequence of mechanical thrombectomy, the technique depicted in FIGS. 7A-7D is more gentle to the vessel walls during the clot removal process. As shown in FIG. 7A a clot C is lodged against the walls of a vessel V. A catheter 300 is introduced into the vessel, as shown in FIG. 7A. As shown in FIG. 7A, In some embodiments, the distal region 305 of the catheter 300 may include openings 309 in a a thrombus interface region 308 that longitudinally overlaps with and/or interfaces with the clot C. In some embodiments, the catheter 300 may additionally include openings 309 in the regions that are proximal and distal to the thrombus interface region 308. During use of a distal region 305 with or without openings, the distal region 305 may be inserted alongside the clot, or through the clot C with the thrombus interface region 308, and the openings 309 thereof, positioned within the clot C, as shown in FIG. 7B. Under applied aspiration via the catheter 300, the clot C can be retrieved into a surrounding catheter 400. With the catheter 300 in place as shown in FIG. 7B, with at least the thrombus interface openings region 308 within or alongside the clot C, an aspiration catheter 400 may be positioned over the catheter 300 so that a distal end of the aspiration catheter 400 interfaces with a proximal end of the clot C, as shown for example in FIG. 7C.

Aspiration of the catheter 300 and/or the aspiration catheter 400 may be implemented with various aspiration schemes. For example, aspiration can include initiating catheter 300 aspiration, then initiating aspiration catheter 400 aspiration, and then withdrawing the catheter/aspiration catheter as a single unit. In some embodiments, aspiration includes initiating aspiration via the aspiration catheter 400, then initiating aspiration via catheter 300, and then withdrawing the catheter/aspiration catheter as a single unit.

Additionally or alternatively, aspiration can include initiating aspiration via both aspiration catheter 400 and catheter 300 simultaneously and then withdrawing the catheter 300 and aspiration catheter 400 together as a single unit. In some implementations, aspiration can include initiating aspiration via the aspiration catheter 400, and then withdrawing both the catheter 300 and the aspiration catheter 400 as a single unit.

In some embodiments, applying aspiration includes initiating aspiration via catheter 300, and then withdrawing both the catheter 300 and the aspiration catheter 400 as a single unit. In such instances, the reduced P_{c} applied to the interior of aspiration catheter 400 via one or more proximal openings, may create a reduced P_{ac} and cause blood clot corking into aspiration catheter.

In various examples, applying aspiration can include initiating aspiration via catheter 300, then advancing the distal end of aspiration catheter 400 to proximal end of clot. Next, aspiration via aspiration catheter 400 can be initiated, followed by withdrawing both the catheter 300 and the aspiration catheter 400 as a single unit.

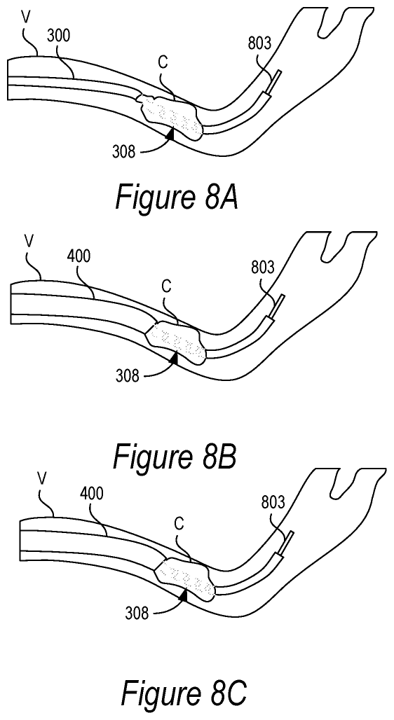

FIGS. 8A-8C show additional and alternative views of removing a clot C with both a catheter 300 and aspiration catheter 400. As depicted in FIG. 8A, the catheter 300 can be introduced through a vessel V of a complex vasculature. A guidewire 803 can be advanced and positioned such that its distal end has crossed the clot. Then the catheter 300 can be slidably advanced over the guidewire 803 and positioned such that the thrombus interface region 308 (which includes a plurality of openings 309) is disposed longitudinally overlapping with and/or engaged with the clot C.

With reference to FIG. 8B, an aspiration catheter 400 can be slidably advanced over the catheter 300, such that the catheter 300 is disposed at least partially within the lumen of the aspiration catheter 400. In the illustrated example, the aspiration catheter 400 is slidably advanced until a distal end of the aspiration catheter 400 abuts the proximal face of the clot C. Aspiration can be supplied via the aspiration catheter 400, thereby adhering the clot C to the aspiration catheter 400 and/or at least partially withdrawing the clot C into the lumen of the aspiration catheter 400. As noted previously, the presence of the catheter 300 can reduce the force of suction required to move the clot C into the aspiration catheter 400, and reduce the risk of clot fragmentation or downstream embolization.

In some implementations, as shown in FIG. 8C, the thrombus interface region 308 can likewise include one or more openings. In such implementations, it may be beneficial to supply suction through the catheter 300 such that the clot C is adhered to the thrombus interface region 308 through the openings therein, instead of or in addition to the aspiration applied via the aspiration catheter 400. In at least some instances, the openings of the thrombus interface region 308 can be in communication with a separate lumen of the catheter 300 that is configured to be fluidly coupled to an extracorporeal suction source. This aspiration lumen of the catheter 300 can be fluidically separated from the lumen that connects the openings of the proximal openings region 306 and the distal openings region 307. In other embodiments, openings across all of the proximal openings region 306, distal openings region 307, and thrombus interface region 308 can all be in communication with a common lumen of the catheter 300.

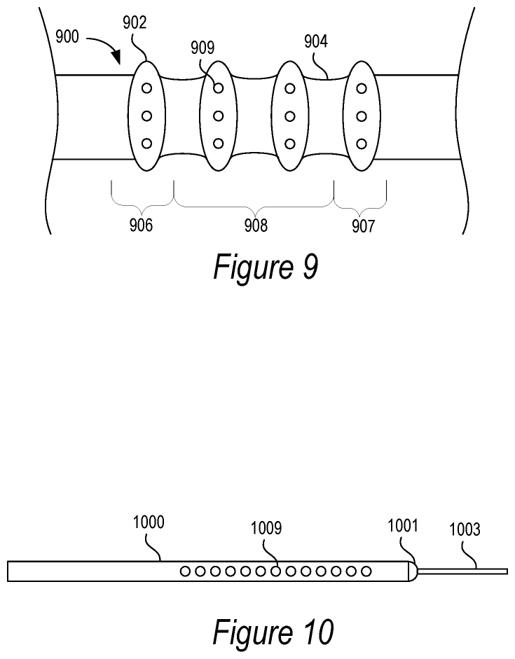

FIG. 9 illustrates an additional example of a catheter 900 in which a proximal openings region 906, a distal openings region 907, and/or a thrombus interface region 908 can include surface features such as ribbing. The openings 909 may be located on the peaks 902 and/or in the valleys 904 of the ribbing. The ribbing provides the advantage of increased engagement and axial force applied to the blood clot during withdrawal. In various examples, the surface features of the catheter 900 can include grooves, ridges, protrusions, recesses, detents, bumps, coil-shaped structure, or any other suitable surface feature. Additionally the openings 909 can be positioned at any suitable location along surface features, including at raised portions (e.g., peaks, bumps, protrusions), or recessed portions (e.g., grooves, indentations, etc.), or both.

As illustrated in FIG. 10, in some embodiments, a catheter 1000 with openings 1009 (similar to openings 309 of the catheter 300 described elsewhere herein) can also include an atraumatic tip permanently affixed at a distal end of the catheter 1000. The atraumatic tip may accommodate placement of the catheter, with or without the need for guidewire 1003. For example, the atraumatic tip can ensure that the catheter 1000 does not puncture the vessel wall while the catheter 1000 is being distally advanced within the vasculature. The atraumatic tip can include a variety of materials. In some embodiments, the atraumatic tip comprises soft materials (e.g., silicone). In some embodiments, the atraumatic tip 1001 includes one or more radiopaque portions or markers (e.g., platinum marker bands, coils, radiopaque filler, or other radiopaque structures). The one or more radiopaque portions can be visualized using fluoroscopy and/or other suitable imaging techniques to assist in positioning the catheter 1000 within a patient's body.

### II. EXAMPLES OF CATHETER SUPPORTING STRUCTURES

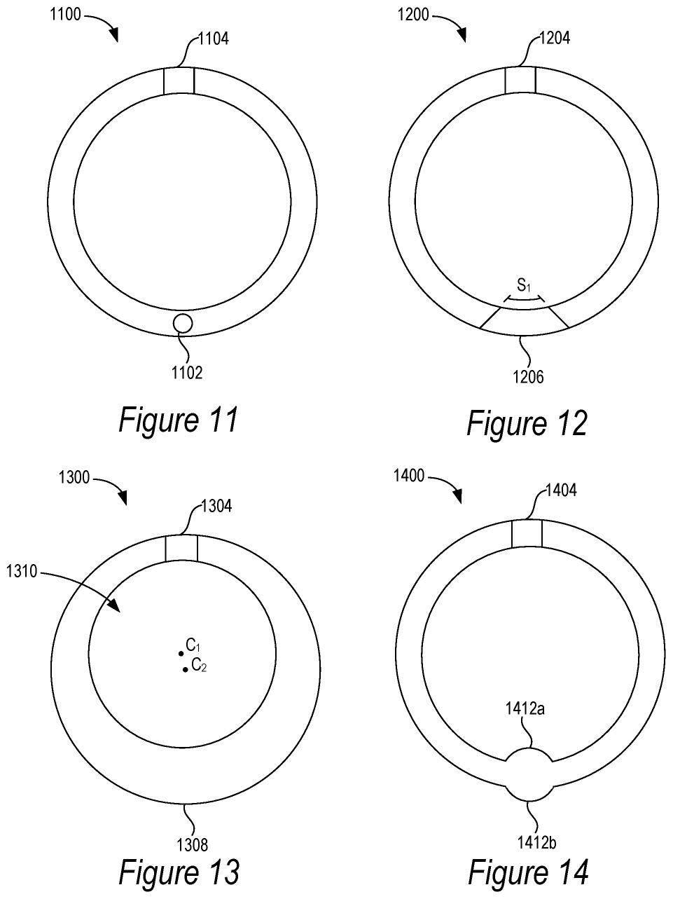

The systems, devices, and methods provided herein can be used in treating a variety of vessel occlusions. In some embodiments, a catheter (e.g., the catheter 300 of FIG. 3) can include one or more additional structural features integrated therein and/or attached thereon that can improve the function of the catheter. For example, in some embodiments, the catheter can include a supporting structure. The supporting structure can be configured to assist the catheter in navigating body lumens. For example, the supporting structure can provide passive support (e.g., stiffen one or more regions of the catheter). Such passive support can beneficially affect movement tendencies of the catheter, for instance by facilitating proximal retraction of the catheter 300 when engaged with a clot in a manner that reduces the risk of clot fragmentation or disengagement. FIGS. 11-17 illustrate a variety of different structural features that can be incorporated into a catheter as described elsewhere herein. In various implementations, these features described below can be combined in any suitable manner, with a single catheter having two or more different structural features along some or all of the length of the catheter.

Referring now to FIG. 11, a cross-section of a catheter 1100 having a supporting structure 1102 and one or more perforations 1104 is shown. The perforations 1104 can be similar to the openings 309 described above with respect to catheter 300. The supporting structure 1102 can serve to stiffen, strengthen, or reinforce the sidewall of the catheter 1100 such that the catheter 1100 is less flexible along the side of the catheter with the supporting structure 1102 than the opposing side that includes the perforation(s) 1104.

The supporting structure 1102 can be an elongate member, for instance taking the form of a wire, fiber, braid, coil, cable, or combination thereof. In some embodiments, the supporting structure 1102 is curved (e.g., sinusoidal, serpentine), curvilinear, or any other suitable geometry. The supporting structure 1102 may generally have a smaller profile than the catheter 1100. In some embodiments, the supporting structure 1102 is substantially coaxial with the catheter 1100. Optionally, the supporting structure 1102 may span the entire length of the catheter 1100.

The supporting structure 1102 can be disposed within a lumen of the catheter 1100 such that the supporting structure 1102 can be moved (e.g., slidably advanced or retracted along a longitudinal direction) relative to the catheter 1100. In such instances, the supporting structure 1102 can be coupled to an external device (not shown) via the proximal end portion of the catheter 1100. For example, the supporting structure 1102 may include one or more coupling elements that couple the supporting structure 1102 to one or more handles. In such cases, the supporting structure 1102 can be manually advanced distally, retracted proximally, and/or rotated. For example, an operator can manipulate the one or more handles to move the supporting structure 1102 through the catheter 1100.

In some embodiments, the supporting structure is a passive supporting structure. For example, a proximal end region of the supporting structure 1102 can be coupled to the catheter 1100 at a proximal region of the catheter 1100 and a distal end region of the supporting structure 1102 can be coupled to the catheter 1100 at a distal region of the catheter 1100. In some embodiments, the supporting structure 1102 only spans a portion of the catheter 1100. For example, the supporting structure 1102 may only span a distal end portion of the catheter 1100. Suitable lengths for the supporting structure 1102 can be 10 mm to 170 mm.

In some embodiments, the supporting structure 1102 is affixed with respect to the catheter 1100 throughout such that the supporting structure 1102 moves in tandem with the catheter 1100. For example, the supporting structure 1102 may rest in a sidewall of the catheter 1100 such that the sidewall of the catheter 1100 is configured to exert one or more forces on the supporting structure 1102 when the catheter 1100 is moved. In response to the one or more forces, the supporting structure 1102 can similarly apply one or more forces to the catheter 1100 so as to influence a movement (e.g., translation, rotation) of the catheter 1100. For example, the supporting structure 1102 can bias the catheter 1100 to move in a direction radially opposite of the supporting structure 1102.

The supporting structure 1102 can include one or more segments. For example, the supporting structure 1102 can include a proximal segment, one or more intermediate segments, and a distal segment. The proximal segment, one or more intermediate segments, and the distal segment can be coupled to one another. In some embodiments, the supporting structure 1102 omits one or more of the proximal segment, one or more intermediate segments, and the distal segment. In some embodiments, the choice of supporting structure segments can be tailored to the patient vasculature so as to improve catheter maneuverability.

The supporting structure 1102 can be formed of known flexible materials, including shape memory and/or superelastic materials (e.g., Nitinol), cobalt, chromium, platinum or other radiopaque materials, stainless steel, other metals or metal alloys, or a combination thereof. In some embodiments, the supporting structure 1102 can be shape set (e.g., heat set) to conform to the vasculature upon deployment of the catheter 1100 at a treatment site. Additionally, or alternatively, the supporting structure 1102 can have a non-uniform thickness throughout. For example, the supporting structure 1102 may have a greater thickness in a proximal portion of the supporting structure 1102 than in a distal portion of the supporting structure 1102. In some embodiments, the supporting structure 1102 has a gradient thickness. For example, the supporting structure 1102 can decrease in thickness from the proximal portion to the distal portion, or vice-versa.

In some embodiments, the supporting structure 1102 is disposed in a sidewall of the elongate body at a position radially opposite to the one or more perforations 1104. For example, as depicted in FIG. 11, the supporting structure 1102 radially opposes the perforations 1104. In some embodiments, the supporting structure 1102 contributes to a stiffness profile of the catheter 1100 such that the catheter 1100 is stiffer in portions surrounding the supporting structure 1102 than the portions surrounding the perforations 1104. Still yet, in some embodiments, the supporting structure 1102 can decrease or leave the stiffness of the catheter 1100 unchanged. For example, the supporting structure 1102 can have a lower stiffness than the rest of the catheter 1100 and be configured to increase the flexibility of the catheter 1100 about the supporting structure 1102.

In some embodiments, the supporting structure 1102 is disposed in a sidewall of the elongate body at a position that is not radially opposite of the perforations 1104. For example, the supporting structure 1102 can be disposed in the sidewall at an angle relative to the perforations 1104 of between 0 to 30 degrees, 30 to 60 degrees, 60 to 90 degrees, 90 to 120 degrees, 150 to 180 degrees, 180 to 210 degrees, etc. In some embodiments, the supporting structure 1102 is placed at an angle relative to the perforations 1104 so as to bias a rotation of the catheter 1100.

Referring now to FIG. 12, a cross-section of a catheter 1200 is shown. The catheter 1200 can have a supporting structure 1206 and one or more perforations 1204. In some embodiments, the supporting structure 1206 is integrally formed with the catheter 1200. The supporting structure 1206 may include one or more radial sections of the catheter 1200. For example, the supporting structure 1206 may span a section S₁ of a sidewall of the catheter 1200. In some embodiments, section S₁ is defined by an arc length that is no more than 10%, 20%, 30%, 40%, 50%, 60%, etc. of an inner circumference of the catheter 1200. In some embodiments, the supporting structure 1206 is formed alongside the catheter 1200. For example, the supporting structure 1206 may be coextruded with the catheter 1200. The supporting structure 1206 can have similar properties to the supporting structure 1102 of catheter 1100 of FIG. 11. For example, the supporting structure 1206 can be configured to exert and/or resist one or more forces relative to the catheter 1200. In some embodiments, the supporting structure 1206 is disposed in a sidewall of an elongate body of the catheter 1200 at a position radially opposite of the perforations 1204.

The supporting structure 1206 can be configured to bias the catheter's 1200 movements toward the perforations 1204. For example, the supporting structure 1206 can increase the likelihood that the catheter 1200 bends or flexes about the perforations 1204 when the catheter is distally advanced and/or proximally retracted. In some embodiments, the supporting structure 1206 causes the catheter 1200 to rotate when the catheter 1200 is navigated through tortuous anatomy. In some embodiments the supporting structure 1206 is radiopaque.

Referring now to FIG. 13, a cross-section of a catheter 1300 is shown. The catheter 1300 can have a supporting structure 1308 and define a non-concentric lumen 1310. The supporting structure 1308 can be integrally formed with the catheter 1300 as a unitary component. For example, the supporting structure 1308 can include a section of the catheter 1300 having a greater thickness than other sections of the catheter 1300. The supporting structure 1308 can include additional material, e.g., for increasing the thickness on one side of the catheter 1300. In some embodiments, the supporting structure 1308 includes additional material formed in the lumen of the catheter 1300, so as to decrease the volume of the lumen of the catheter 1300.

In some embodiments, the lumen of the catheter 1300 can be placed in a non-concentric location relative to the catheter 1300 including the supporting structure 1308. For example, as depicted in FIG. 13, the center C₁ of the lumen 1310 is offset from the center C₂ of the catheter 1300. The relative placement of the supporting structure 1308 can affect the separation between C₁ and C₂ such that the catheter 1300 is biased to move in a certain direction when the catheter is affected by one or more external forces. For example, in some embodiments, the placement of the supporting structure 1308 can cause the catheter 1300 to be biased to move in a direction from C₂ to C₁.

The catheter 1300 can include one or more perforations 1304 that are substantially similar to the perforations 1104 of the catheter 1100 of FIG. 11 and/or the perforations 1204 of the catheter 1200 of FIG. 12. The perforations 1304 can be configured to flex and bend when the catheter 1300 is navigated through body lumens. In some embodiments, the supporting structure 1308 is positioned radially opposite the perforations 1304 and is configured to interact with the perforations 1304 to cause the catheter 1300 to rotate and/or translate.

In some embodiments, the catheter can include one or more ridges, protrusions, bumps, or other such surface features that increase a wall thickness at a defined portion of the sidewall. Referring now to FIG. 14, the catheter 1400 includes one or more ridges 1412. The one or more ridges 1412 can include an interior ridge 1412a. For example, the interior ridge 1412a can protrude into a lumen of the catheter 1400. In some embodiments, the ridges 1412 can include an exterior ridge 1412b. For example, the exterior ridge 1412b can protrude into a lumen of the vessel. The ridges 1412 can include excess material placed on the catheter 1400. In some embodiments, the ridges 1412 include materials similar to the supporting structures previously discussed herein. The ridges 1412 can be configured to influence the movement of the catheter 1400 through body lumens.

In some embodiments, the ridges 1412 provide the catheter 1400 with support and the interior ridge 1412a opposes the exterior ridge 1412b so as to improve the stability of the catheter 1400. The ridges 1412 can include any number of ridges. For example, the ridges 1412 can include 1, 2, 3, 4, 5, 6, 7, 8, 9, 10, 20, 30, or more ridges.

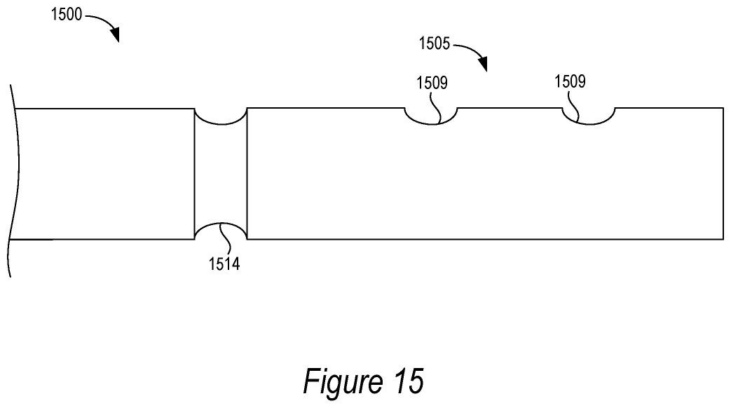

In addition to or instead of the support structures to stiffen a portion of the catheter, in various embodiments a catheter can have a neck region in which the flexibility is increased (i.e., stiffness is reduced) so as to allow for deflection or articulation of the catheter about the neck region. As depicted in FIG. 15, for example, a catheter 1500 can include a distal region 1505 having one or more perforations 1509 as described previously. Proximal to these perforations 1509, the catheter 1500 has a neck region 1514 in which a sidewall thickness of the catheter 1500 is reduced. This can result in increased flexibility about the neck region 1514, such that the distal region 1505 of the catheter 1500 can deflect, bend, or articulate about the neck region 1514, or may allow rotation of the distal region 1505 about the longitudinal axis of the catheter 1500 relative to regions of the catheter 1500 positioned proximal of the neck region 1514. The neck region 1514 can include a portion of the catheter that occupies a smaller radial width than other portions of the catheter (e.g., having a smaller ID and/or smaller OD than adjacent portions of the catheter). For example, the neck region 1514 can occupy a first radial width R₁ that is less than a second radial width R₂ of the catheter 1500. In some embodiments, the first radial width R₁ is no more than 90%, 80%, 70%, 60%, 50%, 40%, 30%, 20%, etc. of R₂. The neck region 1514 can be configured to increase the flexibility of the catheter 1500. For example, the neck region 1514 can provide one or more inflection points of the catheter 1500. In such cases, the catheter 1500 may preferentially bend at the neck region 1514 while the catheter 1500 is maneuvered through body lumens. In some embodiments, the neck region 1514 advantageously allows the catheter 1500 to pass through tortuous anatomy having complex curvatures.

In some embodiments, the neck region 1514 comprises the same material as the rest of the catheter 1500. However, in other embodiments, the neck region 1514 can include one or more materials that are different from the rest of the catheter 1500. For example, the neck region 1514 can include one or more materials that cause the neck region 1514 to have a lower stiffness than the rest of the catheter 1500.

Further, in some embodiments, the neck region 1514 is a separate component from the catheter 1500 altogether. In such cases, the neck region 1514 may be configured to couple a proximal segment of the catheter with a distal segment of the catheter. While the neck region 1514 is depicted as occupying a small section of the catheter 1500, it should be understood that the neck region 1514 can occupy a large portion of the catheter 1500. For example, in some embodiments, the neck region 1514 can span at least 5%, 10%, 20%, 30%, 40%, or 50% of the length of the catheter 1500.

In some embodiments, the neck region 1514 is a first neck region and the catheter 1500 includes additional neck region(s). In some embodiments, having multiple neck regions can provide the advantage of multiple inflection points so as to increase maneuverability of the catheter 1500.

In an embodiment, the catheter 1500, with or without neck region 1514, with openings 1509 only in the thrombus interface region, and a valve at or adjacent its distal tip to seal against a guidewire, is used to remove a clot lodged against the walls of a vessel. In some embodiments, there can be two or more openings 1509. The guidewire can be advanced into the vasculature and positioned such that its distal end has crossed the clot. Then the catheter 1500 can be slidably advanced over the guidewire and positioned such that the thrombus interface region is disposed longitudinally overlapping with and/or engaged with the clot. In this configuration, vacuum applied to catheter 1500 from its proximal end draws part of the clot into the openings in the thrombus interface region, securing the clot to the distal region of catheter 1500. The catheter 1500 is then withdrawn from the anatomy to remove the clot. In some embodiments, the catheter 1500 described above can be used in conjunction with an aspiration catheter 400 to remove the clot, for instance with aspiration being applied to both the aspiration catheter 400 and the catheter 1500.

In an embodiment, a catheter similar to 1500, with or without neck region 1514, with openings 1509 only in the thrombus interface region, and a normally-closed valve at or adjacent its distal tip allowing passage of a guidewire, is used to remove a clot lodged against the walls of a vessel. In various examples, there can be two or more openings 1509. The guidewire can be advanced into the vasculature and positioned such that its distal end has crossed the clot. Then the catheter 1500 can be slidably advanced over the guidewire and positioned such that the thrombus interface region is disposed longitudinally overlapping with and/or engaged with the clot. The guidewire is then removed from the catheter. In this configuration, vacuum applied to catheter 1500 from its proximal end draws part of the clot into the openings in the thrombus interface region, securing the clot to the distal region of catheter 1500. The catheter 1500 is then withdrawn from the anatomy to remove the clot. In some embodiments, the catheter 1500 described above can be used in conjunction with an aspiration catheter 400 to remove the clot, for instance with aspiration being applied to both the aspiration catheter 400 and the catheter 1500.

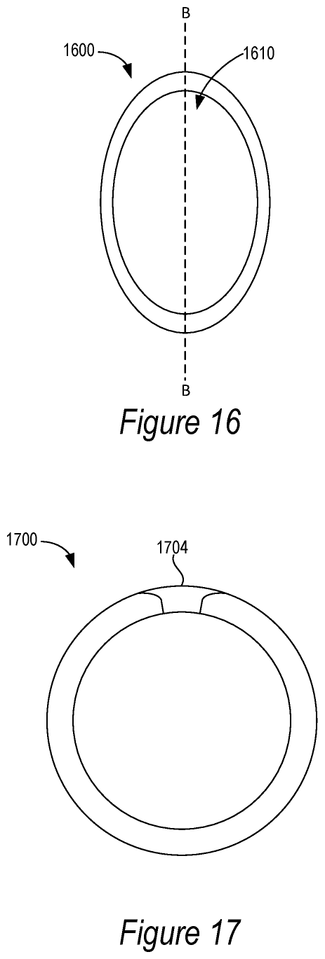

Turning now to FIG. 16, a catheter 1600 having an oval cross-section is shown. The catheter 1600 defines a lumen 1610 that also has an oval cross-section. In some implementations, the cross-sectional shape can be egg-shaped, for instance having a greater inner diameter and/or outer diameter in a lower half of the cross-sectional view than in the upper half of the cross-sectional view. The catheter 1600 can include one or more integrated components that affect the shape of the catheter 1600. For example, the catheter 1600 may have one or more integrated support structures that cause the catheter 1600 to depart from a strictly circular cross-section. For example, one or more integrated support structures may cause the catheter 1600 to be stretched along the axis B-B. In some embodiments, the catheter 1600 is preferentially stretched so as to enhance its maneuverability through tortuous anatomy. For example, in some embodiments, the oval cross-section of the catheter 1600 enables the catheter 1600 to more easily pass through oval-shaped vasculature. An oval cross-section distal region may cross through or alongside the clot more easily than a circular cross-section. An oval cross-section distal region may isolate more of the clot circumference from the vessel wall, resulting in reduced friction of the clot against the vessel wall during removal. An oval cross-section distal region may engage more of the clot circumference, resulting in increased friction of the clot against the distal region during removal. An oval cross-section distal region may allow larger openings, particularly openings in the thrombus engagement region, resulting in larger suction force against the clot when vacuum is applied to catheter 1600. Many variations of the catheter shape are possible. For example, the catheter 1600 can have other oblong cross-sections, substantially rectangular cross-sections, etc. In cases where the catheter 1600 has an unconventional cross-section, the one or more supporting structures can assist the catheter 1600 as it navigates through body lumens. In some embodiments, the catheter can be formed from one or more radiopaque materials. For example, all or portions of the catheter 1600 can include barium sulfate, which produces a radiopacity in the catheter 1600.

The perforations in the catheter can have any variety of shapes. For example, the perforations can include tapered sections and/or indentations. Referring now to FIG. 17, a cross-section of a catheter 1700 having a rounded perforation 1704 is shown. The rounded perforation 1704 covers a greater external arc length than an internal arc length. The rounded perforation 1704 can preferentially cause the catheter 1700 to bend at a certain angle (e.g., 20 degrees) when the catheter is affected by one or more external forces.

The supporting structures and modifications to the catheter disclosed herein can be configured to be compatible with an aspiration catheter. For example, the aspiration catheter can comfortably slide over the catheter and any protrusions and/or ridges caused by supporting structures extending therefrom. In some embodiments, the supporting structures are configured to assist in the placement and/or retraction of the aspiration catheter. For example, the supporting structures can create space and pathways for the aspiration catheter to pass through. In some embodiments, the supporting structures can improve access to the thrombus by causing the catheter to straighten the vessel (e.g., similarly to the straightening process of FIG. 6).

The supporting structures and modifications to the catheter can be used in any of the clot removal methods of the present disclosure. In some embodiments, placement of the supporting structures causes the clot to traverse along a path of least resistance upon aspiration. For example, the supporting structures can be biased to bend about an inside radius of twists and turns in the vasculature, while the clot traverses a pathway away from the inside radius of the twists and turns. In such cases, the clot may be withdrawn more easily through freer, less obstructed pathways and less likely to be detached from the distal region while traversing twists and turns in the vasculature. In embodiments where the supporting structure opposes one or more perforations, for example as in the catheter 1100 of FIG. 11, the clot may naturally traverse a path closer to the perforations 1104 than the supporting structure 1102. As will be understood by someone experienced in the art, many variations and combinations of supporting structures can be employed to allow a clot to traverse the path of least resistance. Furthermore, the same effect may be achieved with the use of perforations alone, such as by reducing the stiffness in one section of the catheter.

### III. CONCLUSION

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) can apply to all configurations, or one or more configurations. Such disclosure can provide one or more examples. A phrase such as an aspect can refer to one or more aspects and vice versa, and this applies similarly to other phrases.

Any implementation described herein as an “example” is not necessarily to be construed as preferred or advantageous over other implementations. Furthermore, to the extent that the term “include,” “have,” or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

Although many of the embodiments are described above with respect to systems, devices, and methods for treating vessel obstructions, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-17.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A method for removing a clot from a vessel with a catheter and an aspiration catheter, wherein the catheter comprises:
an elongate body defining a lumen extending between a proximal region and a distal region of the elongate body,
wherein the distal region comprises a distal openings region, a proximal openings region and a thrombus interface region between the distal openings region and the proximal openings region, and
wherein the distal openings region and the proximal openings region each comprise one or more openings in fluid communication with the lumen,
the method comprising:
positioning the catheter from a proximal side of the clot so that the distal region of the catheter extends through the clot with the distal openings region positioned at least partially distal of the clot and the proximal openings region positioned at least partially proximal of the clot;
positioning an aspiration catheter distal end at the proximal end of the clot; and
withdrawing the clot in the proximal direction with a combination of aspiration applied to one or more of the catheter or the aspiration catheter.

2. The method of claim 1, wherein the catheter is positioned outside of and parallel to the aspiration catheter.

3. The method of claim 1, wherein the aspiration catheter is positioned over the catheter.

4. The method of claim 1, wherein the aspiration catheter and the catheter are integrally formed as a single unit.

5. The method of claim 1, wherein positioning the catheter so that the distal region of the catheter extends through the clot with the distal openings region positioned distal of the clot and the proximal openings region positioned proximal of the clot cause the pressure differential between the proximal side of the clot and the distal side of the clot to be reduced.

6. The method of claim 5, wherein during withdrawing of the clot, the position of the catheter relative to the clot with the distal openings region positioned distal of the clot and the proximal openings region positioned proximal of the clot maintained in order to maintain the reduce pressure differential.

7. The method of claim 1, wherein withdrawing the clot in the proximal direction comprising applying aspiration to both the catheter and the aspiration catheter.

8. The method of claim 7, wherein aspiration is applied first to the catheter and then to the aspiration catheter.

9. The method of claim 7, wherein aspiration is applied first to the aspiration catheter and then to the catheter.

10. The method of claim 7, wherein aspiration is applied simultaneously to the catheter and to the aspiration catheter.

11. The method of claim 7, wherein aspiration is applied to the aspiration catheter and not to the catheter.

12. The method of claim 7, wherein aspiration is applied to the catheter and not to the aspiration catheter.

13. The method of claim 1, wherein the catheter comprises a supporting structure housed in the elongate body.

14. The method of claim 13, wherein the supporting structure is disposed in a sidewall of the elongate body at a position radially opposite one or more openings of one or more of the proximal openings region and the distal openings region.

15. The method of claim 13, wherein the supporting structure has a greater stiffness than the elongate body.

16. The method of claim 1, wherein the elongate body comprises one or more ridges integrally formed thereon.

17. A method for removing a clot from a vessel with a catheter and an aspiration catheter, wherein the catheter comprises:
an elongate body defining a lumen extending between a proximal region and a distal region of the elongate body,
wherein the distal region comprises a thrombus interface region,
wherein the thrombus interface region comprises one or more openings in fluid communication with the lumen,
the method comprising:
positioning the catheter from a proximal side of the clot so that the distal region of the catheter extends through the clot with the openings of the thrombus interface region positioned within the clot;
positioning an aspiration catheter distal end at the proximal end of the clot; and
withdrawing the clot in the proximal direction with a combination of aspiration applied to one or more of the catheter or the aspiration catheter.

18. The method of claim 17, wherein the catheter is positioned outside of and parallel to the aspiration catheter.

19. The method of claim 17, wherein the aspiration catheter is positioned over the catheter.

20. The method of claim 17, wherein the aspiration catheter and the catheter are integrally formed as a single unit.

21. The method of claim 17, wherein positioning the catheter so that the distal region of the catheter extends through the clot with a distal openings region positioned distal of the clot and a proximal openings region positioned proximal of the clot cause the pressure differential between the proximal side of the clot and the distal side of the clot to be reduced.

22. The method of claim 17, wherein withdrawing the clot in the proximal direction comprises applying aspiration to both the catheter and the aspiration catheter.

23. The method of claim 17, wherein the catheter comprises a supporting structure housed in the elongate body.

24. The method of claim 23, wherein the supporting structure is disposed in a sidewall of the elongate body at a position radially opposite one or more openings of one or more of a proximal openings region and a distal openings region.

25. A method for removing a clot from a vessel with a catheter, wherein the catheter comprises:
an elongate body extending between a proximal region and a distal region, the elongate body defining a first lumen having a distal terminus and a second lumen extending to the distal region,
wherein the distal region comprises a thrombus interface region,
wherein the distal region comprises a distal openings region, a proximal openings region and a thrombus interface region between the distal openings region and the proximal openings region,
wherein the distal openings region and the proximal openings region each comprise one or more openings in fluid communication with the second lumen
the method comprising:
positioning the catheter from a proximal side of the clot so that the distal region of the catheter extends through the clot with the openings of the thrombus interface region positioned within the clot and the distal terminus of the first lumen is positioned on a proximal side of the clot; and
withdrawing the clot in the proximal direction with a combination of aspiration applied to one or more of the first lumen or the second lumen.
